(19) United States
(12) Patent Application Publication
(10) Pub. No.: US 2019/0247207 A1
(43) Pub. Date: Aug. 15, 2019

(54) STENT HAVING AN ANCHOR FOR TISSUE INGROWTH

(71) Applicant: Cook Medical Technologies LLC, Bloomington, IN (US)

(72) Inventors: John Crowder Sigmon, JR., Winston-Salem, NC (US); Gregory J. Hardy, Colonie, NY (US); Shaun D. Gittard, Winston-Salem, NC (US)

(21) Appl. No.: 16/273,715

(22) Filed: Feb. 12, 2019

Related U.S. Application Data
(60) Provisional application No. 62/629,332, filed on Feb. 12, 2018.

(51) Int. Cl. A61F 2/90; A61F 2/848; A61L 31/16

(52) U.S. Cl. CPC A61F 2/90; A61B 17/12013; A61L 31/16; A61F 2/848

(57) ABSTRACT

A method may include deploying a stent such that at least one opening of the stent is aligned with a tissue surface within a body of a patient, manipulating a tissue portion through the at least one opening of the stent, and securing the tissue portion with respect to the opening of the stent to prevent migration of the stent. Another general aspect of the present disclosure includes a stent. The stent may have a body including a plurality of struts, a covering extending to an end of the body, the end being a proximal end of a distal end, and an anchor secured to the end of the body, where the anchor includes an opening that is exposed with respect to the covering.

### CROSS REFERENCE TO RELATED APPLICATION

The present application claims the benefit of the filing date under 35 U.S.C. § 119(e) of U.S. Provisional Patent Application Ser. No. 62/629,332, filed Feb. 12, 2018, which is hereby incorporated by reference in its entirety.

### BACKGROUND

Stents are medical devices commonly used to maintain patency of diseased body vessels, such as those of the vascular and gastrointestinal systems. Stents are often delivered via a minimally invasive procedure and thereafter expanded to contact and support the inner wall of the targeted vessel. In general, most stents include a tubular shaped support structure having a plurality of interstices or struts configured to facilitate compression and expansion of the stent.

Esophageal self-expandable metal stent placement is a common procedure used to palliate inoperable esophageal carcinoma, manage benign strictures, seal the esophagus after a perforation, and manage other issues. Stents have achieved positive results in the esophagus, but complications occasionally arise. For example, when a stent is used, migration of the stent is one of the most common complications, arising between about 4-36% of the time depending on certain factors. After a stent migrates, the stent may fail to serve its intended purpose and may cause additional detrimental effects. For example, migrated stents may cause chest pain, recurrent dysphagia, and even more serious effects such as intestinal obstruction. Further, a migrated stent may be difficult to retrieve from the body, particularly without using invasive procedures.

In light of this background, it would be advantageous to provide an improved stent having features for preventing stent migration without inhibiting the stent's primary purpose and without increasing the difficulty and invasiveness of removing the stent after a period of time.

### BRIEF SUMMARY

One general aspect of the present disclosure is a method for installing a stent within a patient body. The method may include deploying the stent such that at least one opening of the stent is aligned with a tissue surface within a body of a patient. The method may further include manipulating a tissue portion through the at least one opening of the stent. The method may further include securing the tissue portion with respect to the opening of the stent to prevent migration of the stent.

Another general aspect of the present disclosure includes a stent. The stent may have a body including a plurality of struts, a covering extending to an end of the body, the end being a proximal end of a distal end, and an anchor secured to the end of the body, where the anchor includes an opening that is exposed with respect to the covering.

Another general aspect of the present disclosure includes a stent with a biodegradable portion. The stent may further include a second portion, where the biodegradable portion is secured to a proximal end or a distal end of the second portion. The stent may further include a stent covering that at least partially covers the second portion of the stent. The biodegradable portion may include at least one opening that is exposed with respect to the cover to facilitate tissue ingrowth when the stent is deployed against a tissue surface.

### BRIEF DESCRIPTION OF THE DRAWINGS

The embodiments of the present disclosure may be better understood with reference to the following drawings and description. The components in the figures are not necessarily to scale, with emphasis instead being placed upon illustrating the principles of the present disclosure. Moreover, in the figures, like referenced numerals designate corresponding parts throughout the different views and arrangements.

FIG. 1 is an illustration showing an embodiment of a stent with anchors for tissue ingrowth in accordance with certain embodiments of the present disclosure.

FIG. 2 is an illustration showing a fully covered stent with anchors for tissue ingrowth in accordance with certain embodiments of the present disclosure.

FIG. 3 is an illustration showing a tissue portion being received by an anchor of a covered stent in accordance with certain embodiments of the present disclosure.

FIG. 4 is an illustration showing a tissue portion being secured to an anchor of a covered stent with a band in accordance with certain embodiments of the present disclosure.

FIG. 5 is an illustration showing a tissue portion secured through a cell of an uncovered portion of a stent in accordance with certain embodiments of the present disclosure.

FIG. 6 is an illustration showing a partial cutaway view of an embodiment of a stent with a biodegradable portion having anchors and cells for tissue ingrowth and a covered second portion in accordance with certain embodiments of the present disclosure.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The invention is described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale, and in certain instances details have been omitted which are not necessary for an understanding of the present invention, such as conventional fabrication and assembly.

As used in the specification, the terms proximal and distal should be understood as being in the terms of a physician delivering the medical device to a patient. Hence the term “distal” means the portion of the medical device that is farthest from the physician and the term “proximal” means the portion of the medical device that is nearest to the physician.

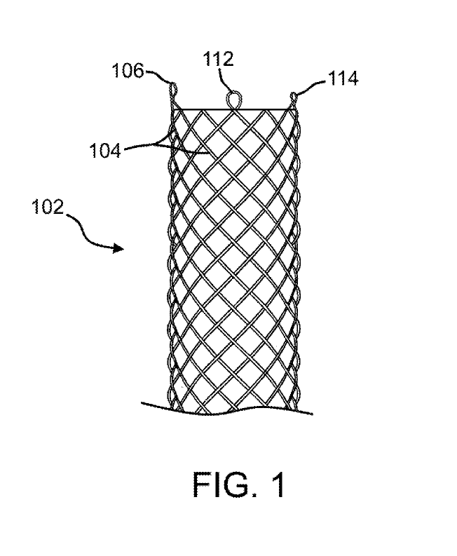

FIG. 1 is an illustration showing an embodiment of a stent 102 with anchors 106, 112, 114 for preventing stent migration (as described in more detail below). In one non-limiting embodiment, the stent 102 may be an esophageal stent for use in the esophagus, but it should be recognized that features described herein may be applied to any other type of stent for use in any suitable location (e.g., a body vessel with a wall or other tissue surface) of a human or animal body. The stent 102 may be self-expanding or may expand under external pressures, for example from an inflatable balloon at the tip of a balloon catheter. The stent 102 may include any suitable stent pattern. One example of a stent pattern is the Z-stent or Gianturco stent design. The stent pattern may include a series of substantially straight segments or struts 104 interconnected by a series of bent segments or bends. The bent segments may include acute bends or apices. The segments may be arranged in a zigzag configuration where the straight segments are set at angles relative to one another and are connected by the bent segments. The stent 102 may alternatively or additionally be formed of another stent pattern, such as an annular or helical stent pattern. Without limitation, the segments mentioned herein may be made from standard medical grade stainless steel or other materials. For example, Nitinol®, a known shape memory alloy including nickel (Ni) and titanium (Ti), may be included in the struts 104.

The stent is depicted as having struts 104 that are uncovered, but in other embodiments (and as described below), the struts 104 may be fully covered or partially covered. If the stent 102 is uncovered (as depicted), the uncovered struts 104 may contact tissue of the esophagus wall when the stent 102 is deployed. Over time (e.g., typically between 3-7 days), the tissue of the esophagus wall may form tissue ingrowth through and/or around the struts 104 so that the stent 102 becomes embedded in the wall of the esophagus. Advantageously, the embedded nature of the stent 102 may substantially prevent it from migrating. However, an uncovered stent may be difficult to remove, and thus it may not be ideal for all procedures.

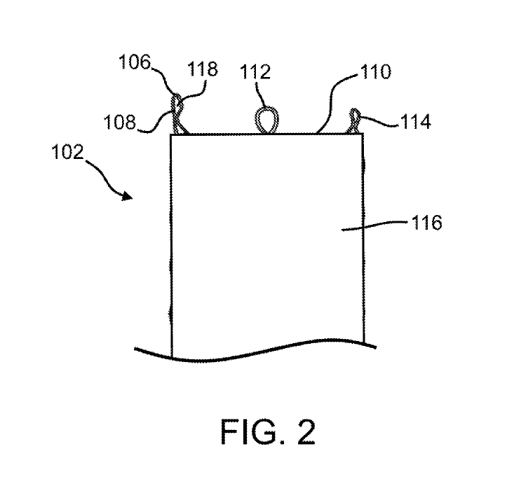

As shown in FIG. 2, in some medical procedures, the stent 102 may be partially or fully covered by a covering 116. The covering 116 may be formed with a silicon material and/or any other suitable material, and may prevent or inhibit tissue ingrowth such that stent 102 may be deployed temporarily and then removed later. However, one disadvantage with common covered stents is that, without tissue ingrowth, covered stents may be prone to migration. In view of this problem, it may be advantageous to use a partially covered stent in some instances to allow for some amount of tissue ingrowth while still making it possible to remove later. However, partially-covered stents may still have an unacceptable migration rate, and even an uncovered stent may have an unacceptable rate of migration before tissue ingrowth takes full effect. Further, it may be difficult to remove partially-covered and uncovered stents due to the uncovered area being coupled to the body of a patient via tissue ingrown.

To solve this problem, the stent 102 may include one or more anchors 106, 112, 114. While many embodiments are contemplated, in some embodiments, the anchor 106 may be formed with a loop 108 of metal wire surrounding an opening 118 (and while a “loop” may be an oval, the term “loop” does not require any particular shape in this disclosure). The loop 108 of metal wire may be integral and continuous with at least one of the struts 104 shown in FIG. 1, or alternatively the anchor 106 may be separately formed and attached to the body of the stent 102. The anchor 106 may be rigidly fixed to at least one strut 104 of the stent 102, or, the anchor 106 may be at least partially movable with respect to the struts 104, which may be advantageous for ensuring a proper fit within the patient and ease of installation.

In the depicted embodiment of FIG. 2, the anchor 106 is located at a proximal end 110 of the stent 102, but other locations are also contemplated. If the loop is circular in shape, the loop 108 of the anchor 106 may be between approximately 5 mm to approximately 25 mm in diameter, such as about 15 mm in diameter. In some embodiments, the loop 108 may be slightly larger than an outer diameter of a cap of an endoscope (described below), which may be advantageous such that the cap can extend through the loop 108 and engage a tissue surface.

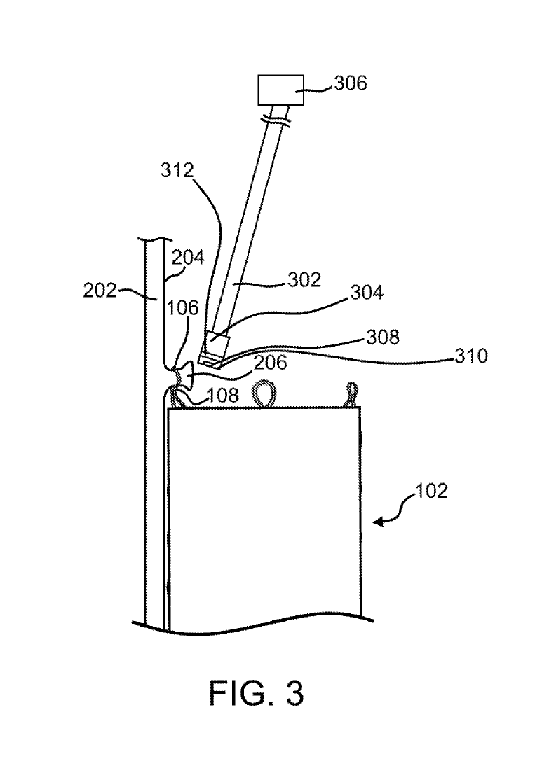

The stent 102 may also include a second anchor 112 and a third anchor 114 as depicted, and it is not limited to three anchors. Each of the anchors 106, 112, 114 may be formed with different dimensions (as shown) and/or different materials, but alternatively, at least two of them may be substantially identical. The anchors 106, 112, 114 may be configured for maximizing tissue ingrowth, and thus, it is contemplated that no additional method for enhancing the tissue ingrowth may be necessary beyond deployment of the stent 102. However, it may be advantageous to enhance tissue ingrowth using the method of engaging a tissue portion 206 described below with reference to FIG. 3.

FIG. 3 is an illustration showing the tissue portion 206 being received by the anchor 106 of the stent 102. As depicted, the tissue portion 206 may be a protrusion of tissue from the outer surface of the wall 204 of an esophagus 202, but other types of tissue may also be used. Receipt of the tissue portion 206 by the anchor 106 may be accomplished by manipulating (e.g., pulling) the tissue portion 206 at least partially through the opening 118 of the anchor 106, which may be performed using any using any suitable device or method.

In the depicted embodiment, after the stent 102 is deployed to a suitable location within the esophagus 202, the manipulation of the tissue portion 206 is accomplished through use of an endoscope 302. In some embodiments, the endoscope 302 may include a gripping device (not shown) and/or may be coupled to a vacuum source 306 to provide a suction force on the tissue portion 206. The suction force may be used to force the tissue portion 206 away from the wall 204 of the esophagus 202 and through the opening 118 of the anchor 106.

For example, initially, the tissue portion 206 may be flush with the remainder of the wall 204 of the esophagus 202 prior to manipulation). Then, a cap 304 of the endoscope 302, which may have an opening 308 at a distal end 310 that communicates with a vacuum source 306, may be placed in a location such that it contacts or in close proximity to the tissue portion 206—and then can pull the tissue portion 206 through the opening 118 upon initiation of the vacuum source 306. If necessary, the distal end 310 of the endoscope may be moved with respect to the loop 108 to guide the tissue portion 206 through the opening 118, but in other embodiments, the vacuum source 306 alone may be sufficient.

Once the tissue portion 206 is pulled through the opening 118 of the anchor 106, a securement device may be used to retain the tissue portion 206 through the opening 118 of the anchor 106. The securement device may be any suitable device (e.g., a mechanical clip or other fastener, a band formed of rubber, a sewn seam of tissue, etc.). In the depicted embodiment, the securement device includes at least one band 312 formed of a material with a high elasticity and resilience. The band 312 may be formed of a natural rubber latex in some embodiments, but other materials are also contemplated (e.g., silicon, urethane, etc.).

The band 312 may be installed around the tissue portion 206 by the endoscope 302. For example, the band 312 may be ejected from the cap 304 while the tissue portion 206 is subjected to suction. The band 312 may be provided and installed by any suitable device or method. In some embodiments, the band 312 is installed around the tissue portion 206 by the endoscope cap 304, which may have a mechanism that disengages with the band 312 in a manner such that the band 312 is ejected or “fired” from the cap 304 to its depicted located around the tissue portion 206. For example, the endoscope 302 may include a trigger cord (not shown) at its proximal end that can be pulled to eject the band from the distal end 310 of the cap 304. In one non-limiting specific example, the endoscope 302 and cap 304 may be included in a device marketed as a 6 Shooter® Universal Saeed® Multi-Band Ligator sole by Cook® Medical. The device may be capable of ejecting any suitable number of bands 312 (e.g., 6 bands, for example). Alternatively, instead of using a cap 304 of an endoscope 302 to suction tissue before banding, a smaller tissue portion may be suctioned through the distal end of a catheter and then a band can be pushed off of the same catheter for securement.

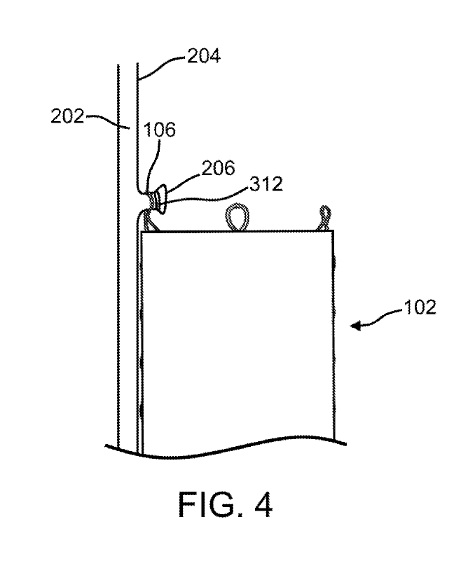

FIG. 4 is an illustration showing the tissue portion 206 when secured by the band 312 and after the above-described endoscope is removed. If left in place, the band 312 may eventually cause tissue necrosis such that the tissue portion 206 sloughs off the wall 204 of the esophagus 202. Thus, the bands may be configured with an appropriate tension around the tissue portion 206 to ensure that enough time passes for sufficient tissue ingrowth around the anchor 106. For example, an appropriate tension of the band 312 may cause the tissue portion 206 to slough off after approximately a week, which may be adequate time for the esophageal tissue to grow into and/or around the anchor 106. Advantageously, after tissue ingrowth occurs at the anchor 106 (or the multiple anchors), the stent 102 may have less of a tendency to migrate with respect to a partially or fully-covered stent without the anchors 106, but the degree of tissue ingrowth may still be low enough that the stent 102 can be removed relatively easily and with a reduced risk of complications and side effects.

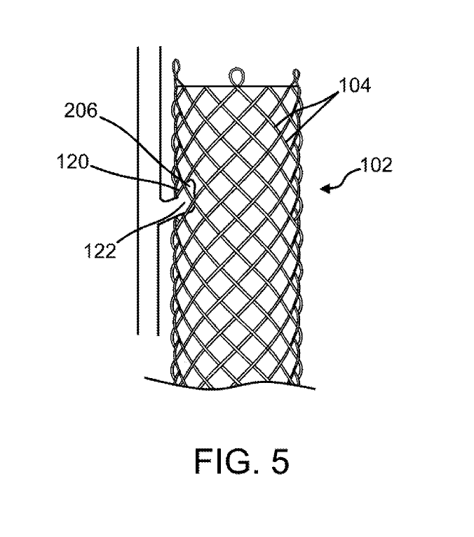

In some embodiments, and as illustrated by FIG. 5, the securement of a stent 102 may additionally or alternatively be enhanced by performing a similar procedure to pull and secure a tissue portion 206 through a cell 120 of a stent 102. The cell 120 of the stent 102 may be formed by struts 104 of the stent 102, and/or separate components may be included to form the cell opening 122. Like the embodiments described above, once the tissue portion 206 is pulled through the cell 120 of the stent 102, the tissue portion 206 may be secured with a band (not shown). Alternatively or additionally, it is contemplated that the struts 104 may have a resilience such that the compression they provide around the tissue portion 206 is adequate, without a band, to hold the tissue portion 206 within the cell opening 122 until adequate tissue ingrowth occurs. For example, it is contemplated that the cell 120 (whether formed by the struts 104 or not) may be formed of a shape memory metal (e.g., Nitinol®) that provides memory retention for enhancing securement of the tissue portion 206. While not shown in FIG. 5, it is further contemplated that a covering may partially or substantially cover the struts 104, but may have an opening corresponding to cells 120 where the above-described tissue ingrowth is desired. These openings of the covering may be cut during installation of the stent or may be pre-fabricated.

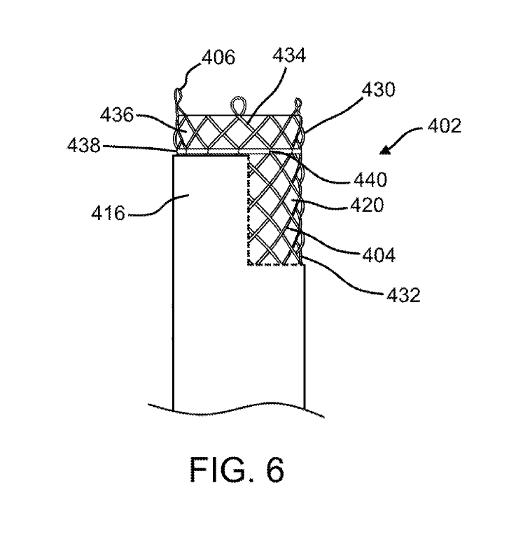

FIG. 6 is an illustration showing a stent 402 with a biodegradable portion 430 and a second portion 432. The embodiment of FIG. 6 may be combined with any of the elements, features, and method steps included with the above-described embodiments. The biodegradable portion 430 may be coupled to a second portion 432, which may have a construction similar or identical to the stent 102 described above (see FIG. 1). For example, referring to FIG. 6, the second portion 432 may include a stent pattern with a series of substantially straight segments or struts 404 made from standard medical grade stainless steel or other materials. Optionally, Nitinol®, a known shape memory alloy including nickel (Ni) and titanium (Ti), may be included in the struts 404. While it is contemplated that the second portion 432 may be uncovered and include cells 420 and/or an anchor (not shown) for tissue ingrowth, the second portion 432 may optionally be covered with the covering 416. In FIG. 6, the covering 416 is only partially depicted (e.g., a portion of the covering 416 is cut away in the view of FIG. 6 such that the second portion 432 is visible).

The biodegradable portion 430 of the stent 402 may be at least partially uncovered, and may be fully uncovered as shown in FIG. 6. In some embodiments, the biodegradable portion 430 may include one or more anchors 406, and said anchors 406 may fully define the biodegradable portion 430. In other embodiments (and as depicted), the biodegradable portion 430 may be formed with a series of braided struts 434 such that the biodegradable portion 430 is expandable when deployed within a human body (e.g., self-expanding or expandable under external pressures such as from an inflatable balloon at the tip of a balloon catheter). The biodegradable portion 430 (e.g., the struts 434 or other structure forming the biodegradable portion 430) may be at least partially formed with a biodegradable material. For example, the struts 434 and/or anchors 406 of the biodegradable portion 430 may be formed with a polydioxanone, a PLGA (poly-lactic-co-glycolic acid), a PGA (poly glycolic acid), a PLLA (poly-L-lactic acid), a polycaprolactone, a polyethylene glycol, and/or any combination/co-polymer blend of these polymers, and/or other suitable biodegradable materials.

Similar to as described above, the anchors 406 and/or the cells 436 of the biodegradable portion 430 may be uncovered such that they are exposed with respect to the covering 316, and thus such that they are capable of facilitating tissue ingrowth when engaged with a tissue surface. In other words, over time (e.g., typically between 3-7 days), the tissue of the esophagus wall (or other tissue surface) may form tissue ingrowth through and/or around the cells 436 and/or the anchors 406 such that the biodegradable portion 430, and therefore the stent 402 itself, are prevented from unintentional migration.

Over time, the biodegradable portion 430 may degrade within the patient body due to its biodegradable material composition. Advantageously, the biodegradable portion 430 may be weakened over a period of weeks, months, or even years, such that, when it is time for the stent 402 to be removed, the weakened biodegradable portion 430 may be broken and/or otherwise decoupled from the second portion 432. After a predetermined period of time, the second portion 432 may then be removed without substantial damage to the esophagus wall or other tissue within the body of the patient.

The biodegradable portion 430 may be “tuned” or configured for stent removal after a particular time period. For example, dimensions of the components of the biodegradable portion 430 (e.g., the thickness of the struts 434) and the specific materials used in their construction may be preselected based on the desired time between stent deployment and removal. In some embodiments, for example, the materials and dimensions of the biodegradable portion 430 may be selected and used such that the biodegradable portion 430 is ready to suitably facilitate removal of the stent 402 after approximately a week from the time of deployment and installation. In other embodiments, the biodegradable portion 430 may be constructed with dimensions and materials such that it retains suitable structural integrity to prevent stent migration for a much longer time period (e.g., up to 6 months, a year, or even longer). It is preferable for the biodegradable portion 430 to be constructed such that it will not completely degrade prior to when the stent 402 will be removed from the body.

Optionally, the biodegradable portion 430 may include a selected failure point 438, where the biodegradable portion 430 is designed to break at the failure point 438 as the stent 402 is removed. The failure point 438 may be the point of attachment between the biodegrade portion 430 and the second portion 432, but other locations are also possible. The failure point 438 may be constructed using dimensions and/or materials such that, at least after a period of biodegradation with the patient, the failure point 438 will be the first location to fracture during stent removal. For example, the failure point 438 may have one or more strands or connectors 440 that have a thickness less than a thickness of the struts 434, a material that degrades faster than the material of the struts 434, or a combination of the two. Advantageously, the failure point 438 may ensure the stent 402 can be cleanly removed without leaving undesirable portions of the stent 402 within the body without creating pointed or jagged edges that may damage body tissue during or after stent removal.

When the biodegradable portion 430 includes the failure point 438, it is contemplated that the remainder of the biodegradable portion 430 may be formed of materials that do not substantially degrade within the patient body. However, it may still be advantageous for the remainder of the biodegradable portion 430 to degrade since it may be left within the patient body after stent removal. Thus, after a period of time, the biodegradable portion 430 may completely degrade, advantageously leaving the patient without an artificial device at the treated area.

In some embodiments, instead of (or in addition to) the inclusion of a biodegradable failure point as described above, the biodegradable portion 430 may be connected to the second portion 432 by another suitable device that is capable of releasing the biodegradable portion 430 from the second portion 432 such that the second portion 432 of the stent 402 can be removed from the patient. For example, a wire or other connector may secure the biodegradable portion 430 to the second portion 432, and the wire or other connector may be cut and/or pulled from the stent 402 by a medical professional during a removal procedure. Additionally or alternatively, a connector may be used that may be weakened and/or fracture by the application of RF energy, the application of an electrical current (e.g., D.C. current), the application of a chemical selected to dissolve the material of the connector, etc.

The figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

We claim:

1. A method comprising:
deploying a stent such that at least one opening of the stent is aligned with a tissue surface within a body of a patient;
manipulating a tissue portion through the at least one opening of the stent; and
securing the tissue portion with respect to the at least one opening of the stent to prevent migration of the stent.

2. The method of claim 1, wherein the tissue portion is pulled through the at least one opening of the stent using an endoscope coupled to a vacuum source, and wherein the vacuum source provides a suction force on the tissue portion.

3. The method of claim 1, wherein securing the tissue portion with respect to the at least one opening of the stent includes placing at least one band at least partially around the tissue portion.

4. The method of claim 3, further comprising deploying the band around the tissue portion by ejecting the band from a cap that is placed onto the end of the endoscope.

5. The method of claim 1, wherein the at least one opening is located within an anchor of the stent, and wherein the anchor is coupled to a proximal end or a distal end of the stent.

6. The method of claim 1, wherein the at least one opening is located within a cell formed by struts of the stent.

7. The method of claim 1, wherein the stent includes a biodegradable portion and a second portion, and wherein the method further comprises removing the second portion from the patient after a predetermined period of time.

8. The method of claim 7, wherein removal of the second portion includes fracturing a failure point of the biodegradable portion to release the biodegradable portion from the second portion.

9. The method of claim 1, wherein the tissue surface is a wall of an esophagus.

10. A stent, comprising:
a body including a plurality of struts;
a covering extending to an end of the body, the end being a proximal end of a distal end; and
an anchor secured to the end of the body, wherein the anchor includes an opening that is exposed with respect to the covering.

11. The stent of claim 10, further comprising:
a biodegradable stent portion and a second stent portion,
wherein the second stent portion includes the body, and
wherein the biodegradable portion includes the anchor and is secured to the end of the body.

12. The stent of claim 10, wherein the anchor is formed with a loop that is continuous with at least one of the struts of the stent.

13. The stent of claim 10, wherein the anchor is movably secured to the proximal end of the body.

14. The stent of claim 10, wherein the anchor includes a shape memory metal.

15. The stent of claim 10, further comprising a second anchor secured to the proximal end of the body, the second anchor having a second opening that has at least one dimension being different than a corresponding dimension of the opening of the anchor.

16. The stent of claim 10, wherein the opening has a diameter of approximately 5 mm to approximately 25 mm.

17. A stent, the stent comprising:
a biodegradable portion;
a second portion, wherein the biodegradable portion is secured to a proximal end or a distal end of the second portion; and
a stent covering that at least partially covers the second portion of the stent,
wherein the biodegradable portion includes at least one opening that is exposed with respect to the cover to facilitate tissue ingrowth when the stent is deployed against a tissue surface.

18. The stent of claim 17, wherein the biodegradable portion includes a failure point formed of at least one biodegradable material such that, after a predetermined period of time within a patient body, the failure point fractures during stent removal.

19. The stent of claim 18, wherein the failure point includes a strand connecting the biodegradable portion to the second portion.

20. The stent of claim 17, wherein the biodegradable portion is constructed with at least one of a polydioxanone, a PLGA (poly-lactic-co-glycolic acid), a PGA (poly glycolic acid), a PLLA (poly-L-lactic acid), a polycaprolactone, and a polyethylene glycol.
